(19) United States
(12) Patent Application Publication
(10) Pub. No.: US 2015/0059968 A1
(43) Pub. Date: Mar. 5, 2015

(54) DEVICE AND METHOD FOR PRINTING FUNCTIONAL MATERIAL ON BIOCOMPATIBLE THIN-FILM

(71) Applicant: Panasonic Intellectual Property Management Co., Ltd., Osaka (JP)

(72) Inventors: MASAYO SHINODA, Tokyo (JP); TOMOKI MASUDA, Osaka (JP); HARUKA KUSUKAME, Nara (JP); MITSURU HARADA, Osaka (JP)

(21) Appl. No.: 14/534,113

(22) Filed: Nov. 5, 2014

Related U.S. Application Data
(63) Continuation of application No. PCT/JP2014/000847, filed on Feb. 19, 2014.

(30) Foreign Application Priority Data
Mar. 1, 2013 (JP) 2013-040471

(51) Int. Cl. B32B 37/00; A61K 8/02; A61Q 1/02; B32B 37/14

(52) U.S. Cl. CPC B32B 37/0046; B32B 37/025; B32B 37/144; A61K 8/0204; A61Q 1/02; B32B 2307/70; B32B 2555/00; A61K 2800/805; A61K 2800/10
USPC 156/240; 156/381

(57) ABSTRACT

A device for printing a functional material on a biocompatible thin-film is provided, including: a feeder for feeding a thin-film sheet including the biocompatible thin-film and a first support into the printing device; a controller for loading makeup information for printing the functional material on the thin-film; a printing unit for printing the functional material corresponding to the makeup information on the thin-film based on a makeup signal from the controller; a feeder for feeding a second support into the device; a transfer unit for transferring a thin-film printed body, the thin-film on which the functional material is printed by the printing unit from the first support to the second support; and a delivery unit for delivering the transferred thin-film printed body to outside the device. This configuration enables people who lack sufficient makeup knowledge, techniques, or time to apply makeup easily, quickly, and safely.

### TECHNICAL FIELD

The present disclosure relates to a device and a method for printing a functional material used, for example, in cosmetic and medical fields on a biocompatible thin-film.

### BACKGROUND ART

In recent years, methods for facial makeup (hereinafter simply referred to as “makeup”) have diversified. This makes it difficult, particularly for people who do not have sufficient knowledge about makeup, to select appropriate makeup from countless options. This is because it takes an enormous amount of time and effort to actually try every makeup to determine and compare the makeup.

In view of the above, for example, PTL 1 and PTL 2 disclose techniques for generating and presenting a simulation image of a face when makeup is applied. Each of the techniques (hereinafter referred to as the “conventional technique”) disclosed in these patent literatures acquires an image by photographing a face to which makeup is to be applied. The conventional technique generates a simulation image by superimposing onto the acquired image an image that shows a makeup condition when lipstick, blush (rouge), and the like are applied to the face. Each of these conventional techniques then displays the generated simulation image on a display device.

Based on the display image, a user can determine suitability of the makeup without actually applying the makeup for comparison and consideration, thereby allowing selection of appropriate makeup with less time and effort.

However, even when makeup suitable for oneself is proposed with such a simulation image, actually applying the makeup to oneself needs a specific cosmetic agent and a makeup set, and makeup techniques and makeup time of some extent or more.

Accordingly, some users may face difficulty in applying the proposed makeup to themselves or may have to take trouble to request a professional beautician or the like to apply the makeup.

The present disclosure therefore provides a device for printing a functional material based on makeup information, such as a cosmetic agent, on a biocompatible thin-film as an effective method for outputting a proposal result made by such a simulation device.

That is, the present disclosure provides a device and a method for printing a functional material on a biocompatible thin-film that allows simple application of makeup customized for an individual and the latest makeup to a human body by printing the functional material based on makeup information on the biocompatible thin-film, and by affixing the biocompatible thin-film on the human body.

### CITATION LIST

### Patent Literatures

PTL 1: Unexamined Japanese Patent Publication No. 2001-346627

PTL 2: Unexamined Japanese Patent Publication No. 2003-44837

### SUMMARY

The present disclosure is directed to a device for printing a functional material on a biocompatible thin-film, the device including: a thin-film sheet feeder for feeding a thin-film sheet including the biocompatible thin-film and a first support into inside of the device for printing the functional material on the biocompatible thin-film; a controller for loading makeup information for printing the functional material on the biocompatible thin-film; a printing unit for printing the functional material corresponding to the makeup information on the biocompatible thin-film based on the makeup information from the controller; a second support feeder for feeding a second support into inside of the device for printing the functional material on the biocompatible thin-film; a transfer unit for transferring a thin-film printed body that is the biocompatible thin-film on which the functional material is printed by the printing unit from the first support to the second support; and a thin-film printed body delivery unit for delivering the thin-film printed body transferred to the second support to outside of the device for printing the functional material on the biocompatible thin-film.

Such a configuration enables, for example, people who do not have sufficient knowledge or techniques about makeup and people who cannot take time for makeup to apply makeup easily, quickly, and safely without assistance.

### BRIEF DESCRIPTION OF DRAWINGS

FIG. 1 is a diagram illustrating an external appearance of a printing device according to a first exemplary embodiment of the present disclosure.

FIG. 2 is a diagram illustrating an internal structure of the printing device according to the first exemplary embodiment of the present disclosure.

FIG. 3 is a diagram illustrating a configuration of the printing device according to the first exemplary embodiment of the present disclosure.

FIG. 4A is a diagram illustrating an operation of the printing device according to the first exemplary embodiment of the present disclosure.

FIG. 4B is a diagram illustrating an operation of the printing device according to the first exemplary embodiment of the present disclosure.

FIG. 4C is a diagram illustrating an operation of the printing device according to the first exemplary embodiment of the present disclosure.

FIG. 4D is a diagram illustrating an operation of the printing device according to the first exemplary embodiment of the present disclosure.

FIG. 4E is a diagram illustrating an operation of the printing device according to the first exemplary embodiment of the present disclosure.

### DESCRIPTION OF EMBODIMENT

### Findings Underlying the Present Disclosure

The present disclosure provides a printing device and a printing method for allowing simple application of makeup customized for an individual and the latest makeup to a human body by printing a functional material based on makeup information, such as a cosmetic agent, on a biocompatible thin-film medium, and by affixing the biocompatible thin-film medium on the human body.

This enables, for example, people who do not have sufficient knowledge or techniques about makeup and people who cannot take time for makeup to apply makeup easily, quickly, and safely.

However, the following problems may be considered in production of a thin-film printed body that is a biocompatible thin-film on which the functional material is printed.

First, since the thin-film itself is directly affixed on the human body, a biocompatible material that does not stimulate or damage the human body needs to be used. In addition, the thin-film is required to have an adhesive property such that the thin-film comes into intimate contact with skin even if an adhesive agent or the like is not laminated.

The thin-film printed body is required to have higher adhesive property of the biocompatible thin-film to the human body to allow good transfer to the skin, that is, to satisfy a relationship of “an adhesive force B<an adhesive force C” to be described later, whoever uses the thin-film printed body.

In order to reduce the makeup coming off due to sebum or the like and in order to allow the cosmetic agent to last long, preferably a thin-film side of the thin-film printed body is brought into intimate contact with a human body side.

Furthermore, since the biocompatible thin-film has a thickness of the order of nanometers, which is so thin that the user does not feel the thin-film is affixed on the skin, handling requires greatest care. For example, the thickness of the nanometer order causes the thin-film to dry easily. Once the thin-film dries, pliability of the thin-film decreases. Therefore, when the thin-film in a dry state absorbs moisture, for example, the pliability increases. However, since rigidity of the thin-film itself is low, it is very difficult to retain the thin-film in a plane state. There is another method of retaining the plane state of the thin-film by, for example, making the thin-film drift in a solution, which however imposes an inconvenience on affixation of the film.

Furthermore, since films to be affixed on skin have previously been thick, such as a sheet-shaped pack cosmetic agent and an adhesive plaster, it is possible to perform position adjustments on the skin as needed after affixation on the skin. However, for a thin-film that is so thin that the user does not feel the thin-film is affixed on the skin, it is difficult to manipulate the film after affixation on the skin.

Therefore, a printing device for producing a thin-film printed body is required to consider humidification for facilitating handling of the thin-film, and to consider secure transfer to the skin.

That is, in the present disclosure, in view of careful handling and good transfer of a cosmetic agent or the like required because a functional material, such as a cosmetic agent, is to be printed on a thin, delicate biocompatible thin-film medium, and in view of safety required because a thin-film printed body is to be affixed on a human body, a printing device has been created that allows printing of a functional material, such as a cosmetic agent, on a biocompatible thin-film.

An exemplary embodiment of the present disclosure will be described in detail below with reference to the drawings.

### First Exemplary Embodiment

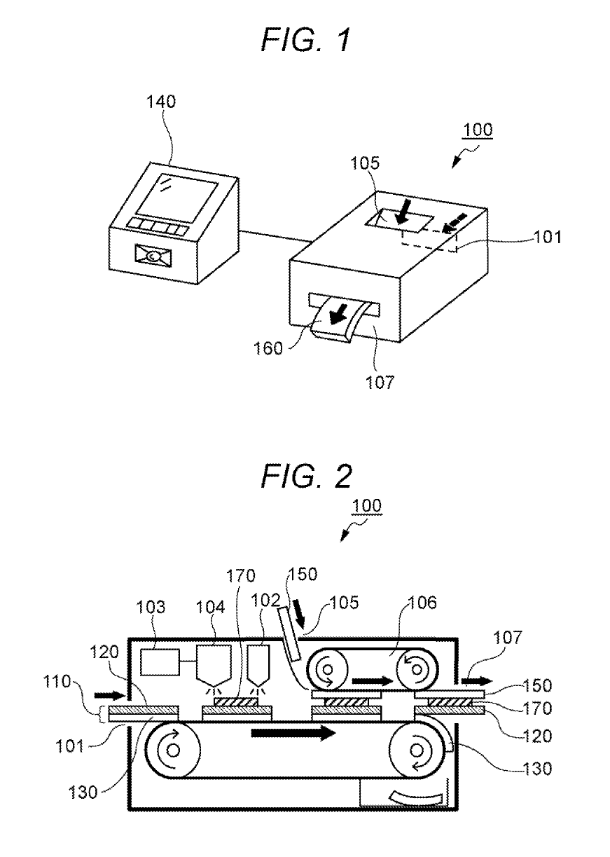

FIG. 1 is a diagram illustrating an external appearance of printing device 100 according to a first exemplary embodiment of the present disclosure. Printing device 100 is connected to makeup information terminal 140, and can receive makeup information. Here, makeup information terminal 140 may include an input unit capable of receiving makeup information related to a user, a camera capable of photographing the user oneself, and the like. In addition, makeup information terminal 140 may be connected to a network to obtain makeup information from outside.

FIG. 2 is a diagram illustrating an internal structure of printing device 100 according to the first exemplary embodiment of the present disclosure. FIG. 2 schematically illustrates thin-film sheet 110 fed from thin-film sheet feeder 101 being delivered from thin-film printed body delivery unit 107 as thin-film printed body 160.

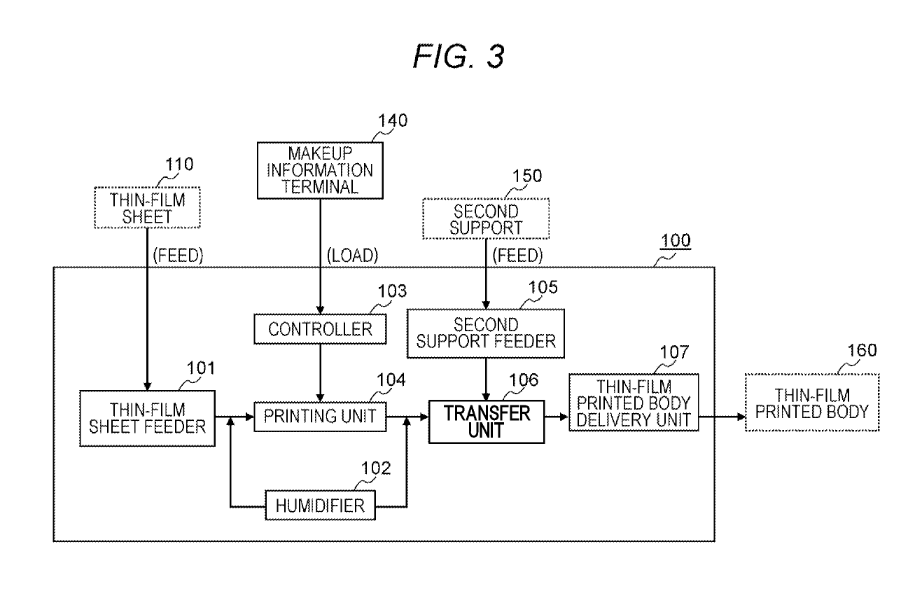

With reference to FIG. 3, a description will now be given of a configuration of printing device 100 according to the first exemplary embodiment of the present disclosure. FIG. 3 is a diagram illustrating the configuration of printing device 100 according to the first exemplary embodiment of the present disclosure.

Printing device 100 includes thin-film sheet feeder 101, humidifier 102, controller 103, printing unit 104, second support feeder 105, transfer unit 106, and thin-film printed body delivery unit 107.

Thin-film sheet feeder 101 includes a roller and drive mechanism for feeding thin-film sheet 110 from outside of printing device 100 into inside of printing device 100.

Here, thin-film sheet 110 is biocompatible thin-film 120 mounted on first support 130 in advance, thin-film 120 serving as a base for printing a functional material, such as a cosmetic agent. Thin-film sheet 110 is provided, for example, in a form of a seal or a roll.

In such thin-film sheet 110, an interface (A) between biocompatible thin-film 120 and first support 130 is retained by, for example, static electricity, chemical adsorption, and physical adsorption. A surface of first support 130 of thin-film sheet 110 has, for example, a hydrophilic group.

Thin-film sheet feeder 101 rolls and feeds thin-film sheet 110 into inside of printing device 100 while rotating a roller that comes in contact with only a first support part of thin-film sheet 110 to avoid damaging biocompatible thin-film 120 of thin-film sheet 110.

Here, biocompatible thin-film 120 in thin-film sheet 110 is required to have a characteristic suitable for print of functional materials such as a cosmetic agent and a medical material, and for subsequent affixation on the human body. In other words, the thin-film itself is a biocompatible, safe material that does not have adverse health effects on the human body, such as a stimulus and a hazardous property.

Preferably, a surface of biocompatible thin-film 120 on a side on which functional material 170 is printed fixes functional material 170 or the like well, and does not allow a component of functional material 170 or the like to penetrate to a surface on an opposite side that is to be affixed onto the human body. This minimizes damage caused by the cosmetic agent or the like to the human body.

In addition, a surface of biocompatible thin-film 120 to be affixed on the human body has an adhesive property capable of coming into intimate contact with skin by itself without the need for another material, such as an adhesive agent. This makes it possible to prevent damage caused by the adhesive agent or the like to the human body, to eliminate a process of adding an adhesive layer to biocompatible thin-film 120, and to facilitate manufacturing of thin-film printed body 160.

Since biocompatible thin-film 120 supports functional material 170, such as a printed cosmetic agent, thin-film printed body 160 including biocompatible thin-film 120 and functional material 170 is adapted to be transferred to skin by being pressed against the skin.

As such a thin-film, for example, a nano thin-film or the like based on a nano sheet of a biocompatible polymer can be used.

Humidifier 102 has a mechanism for humidifying thin-film sheet 110. Humidifier 102 can improve pliability of biocompatible thin-film 120 and lower an adhesive force of the interface (A) between first support 130 and thin-film 120 by, for example, spraying vapor or water-soluble liquid, or by putting thin-film sheet 110 in water or water-soluble liquid. Here, water (vapor) and water-soluble liquid are used because of safety for the human body and being non-stimulative to skin. Furthermore, it is also possible to use water (vapor) and water-soluble liquid with an intention of making the water more antiseptic or maintaining a wet condition of the thin-film for a long time by adding another component into the water-soluble liquid.

Humidifier 102 is, for example, humidifier 102 maintained at constant humidity, and humidifies biocompatible thin-film 120 by putting thin-film 120 in a constant humidity environment. Alternatively, humidifier 102 humidifies thin-film 120 by causing a base material containing water or water-soluble liquid to come into contact with biocompatible thin-film 120.

Humidifier 102 may be provided before or after printing, or both before and after printing in printing unit 104. However, it is possible to obtain an effect of the present exemplary embodiment even if humidifier 102 is not provided.

For example, at a time of printing functional materials that have poor compatibility with water or water-soluble liquid and are difficult to print when the above-described component exists (is easy to print and fix in a dry state), such as a cosmetic agent and a medical material, humidification is preferably performed by humidifier 102 after printing.

When thin-film printed body 160 is not transferred to skin immediately after production, it is necessary to maintain thin-film printed body 160 within printing device 100. As described above, thin-film printed body 160 is easy to dry because of the thickness of the order of nanometer. Accordingly, when thin-film printed body 160 is not transferred to the skin immediately, humidifier 102 can be used for humidifying again the thin-film that has dried with lowered pliability just before thin-film printed body 160 is transferred to the skin. This allows a user to use, print, and transfer thin-film printed body 160 at any desired time without time constraints.

Controller 103 generates printing information about how functional material 170 or the like is to be printed on biocompatible thin-film 120. Controller 103 includes a communication unit for communication with, for example, external makeup information terminal 140, a signal processor, a storage unit, and the like. The generated printing information is sent to printing unit 104 as a printing signal (control signal for a printing operation of a head).

Makeup information terminal 140 with which controller 103 communicates provides makeup information that serves as a base for generating the printing information. The makeup information is, for example, information that shows details of makeup proposed for a user who wants to receive makeup assistance. The makeup information is provided, for example, as an application in makeup information terminal 140 or as cloud service from a server to which makeup information terminal 140 is connected.

Printing unit 104 has a head mechanism for printing functional material 170, such as a cosmetic agent, on biocompatible thin-film 120 of thin-film sheet 110 based on the printing signal from controller 103. Printing unit 104 employs a known printing method, for example, a bubble jet method and a thermal dye-sublimation method. The printing method is not limited as long as functional material 170, such as a cosmetic agent, can be printed well on biocompatible thin-film 120.

Second support feeder 105 includes a roller and drive unit for supplying second support 150 from outside of printing device 100 into inside of printing device 100. Here, second support 150 is a support on which thin-film printed body 160 is transferred from first support 130, and is provided in advance in a form of a seal or a roll. A surface of second support 150 may, for example, receive surface treatment for increasing hydrophilic properties, have an uneven pattern, or use a temperature-responsive polymer. A concentration of either positive or negative may be increased by applying an electric field or the like.

Transfer unit 106 transfers thin-film printed body 160 from first support 130 to second support 150 without damage to thin-film printed body 160, thin-film printed body 160 having makeup information printed by printing unit 104 as functional material 170. Transfer unit 106 includes, for example, a roller and a drive mechanism. At this time, transfer unit 106 transfers thin-film printed body 160 so that a surface of thin-film printed body 160 on which functional material 170 is printed comes into contact with second support 150.

Thin-film printed body delivery unit 107 delivers thin-film printed body 160 produced in transfer unit 106 to outside of printing device 100 as needed. Thin-film printed body delivery unit 107 includes a roller and a drive unit. For example, thin-film printed body delivery unit 107 may be configured to deliver sheet-shaped thin-film printed body 160 in response to a user request to allow the user to affix thin-film printed body 160 by oneself. Alternatively, thin-film printed body delivery unit 107 may have a mechanism in which thin-film printed body delivery unit 107 itself is detachable and thin-film printed body 160 can be transferred directly from thin-film printed body delivery unit 107 to skin by pressing thin-film printed body 160 against a face of a person.

In addition, printing device 100 may include a cutting unit (not illustrated) before thin-film printed body delivery unit 107, the cutting unit being for cutting biocompatible thin-film 120 or thin-film printed body 160 into a desired shape. For example, it is possible to assist simpler makeup by processing thin-film printed body 160 for local makeup, such as rouge and eye shadow, into a shape convenient for transfer to a makeup position.

Furthermore, printing unit 104 may also include a tank (not illustrated) for supplying functional material 170 to be printed on biocompatible thin-film 120. For example, when an ink-jet printing unit is employed, the tank can retain and supply a liquid functional material. Similarly, printing unit 104 may also include a ribbon (not illustrated) for retaining functional material 170 to be transferred to biocompatible thin-film 120. For example, when a thermal transfer printing unit is employed, the ribbon can retain and supply a powdered functional material.

With the foregoing configuration, when thin-film printed body 160 generated according to the present exemplary embodiment is affixed on a human body, a cosmetic agent printed based on the makeup information is easily affixed on skin to produce a visual effect as if makeup has been applied. This enables people who do not have sufficient knowledge or techniques about makeup and people who cannot take time for makeup to apply makeup easily, quickly, and safely.

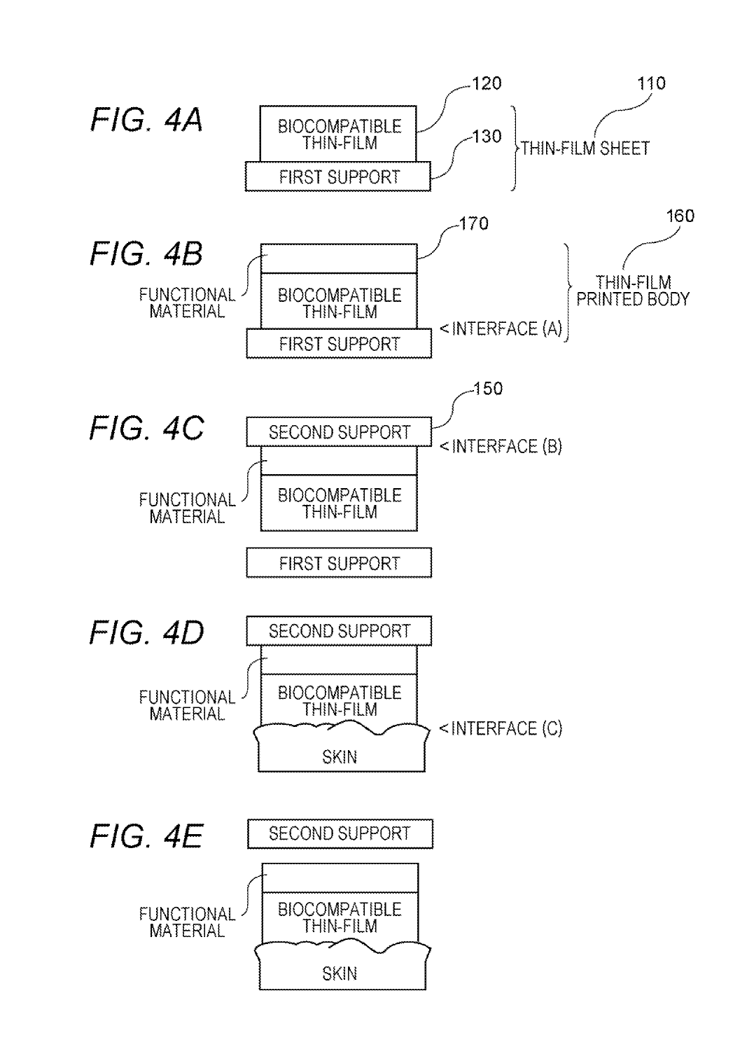

Next, operations of printing device 100 according to the first exemplary embodiment of the present disclosure will be described with reference to FIG. 3 and FIG. 4A to FIG. 4E.

FIG. 4A to FIG. 4E are diagrams each illustrating the operation of printing device 100 according to the first exemplary embodiment of the present disclosure.

First, thin-film sheet feeder 101 supplies thin-film sheet 110 from outside of printing device 100 to inside of printing device 100.

Next, thin-film sheet 110 including biocompatible thin-film 120 and first support 130 is sent to printing unit 104.

Here, printing unit 104 prints functional material 170 corresponding to makeup information from outside on biocompatible thin-film 120 in accordance with printing information generated by controller 103.

At this time, controller 103 communicates with, for example, makeup information terminal 140 that is outside of printing device 100 to obtain the makeup information. Controller 103 generates the printing information about what printing is to be made on biocompatible thin-film 120 in order to apply the makeup, and transmits the printing information to printing unit 104 as a control signal.

Based on the control signal, printing unit 104 drives a head to print functional material 170 on biocompatible thin-film 120.

As a result, thin-film printed body 160 is formed with functional material 170 laminated on biocompatible thin-film 120.

As functional material 170, it is possible to select not only a cosmetic agent for female makeup but also every material that can perform functions required in medical or other fields.

In addition, preferably a thickness of biocompatible thin-film 120 is between 10 nm and 10,000 nm inclusive (between 10 nm and 10 μm inclusive). Particularly, a thickness of between 10 nm and 500 nm inclusive is more preferable. This is because a test result shows that the adhesive property to skin is good when the thickness is 500 nm or less.

Furthermore, it is possible to use an arbitrary material as a material for biocompatible thin-film 120 with no particular limitation. Examples of the materials for the thin-film include polyesters represented by polyglycolic acid, polylactic acid, polycaprolactone, polyethylene succinate, and polyethylene terephthalate, or copolymers thereof; polyethers represented by polyethylene glycol and polypropylene glycol; polyamides represented by nylon, polyglutamic acid, and polyaspartic acid, or salts thereof; polysaccharides represented by pullulan, cellulose, starch, chitin, chitosan, alginic acid, hyaluronic acid, and cornstarch, or salts thereof; silicones represented by acrylic silicone and trimethylsiloxysilicate; acrylic acids represented by alkyl acrylate, silicone acrylate, and amide acrylate, or copolymers thereof; polyvinyl alcohol; polyurethane; polycarbonate; polyacid anhydride; polyethylene; and polypropylene.

Here, the makeup information means information for printing a material that supports some functional components such as components for wide variety of uses including cosmetic and medical uses on biocompatible thin-film 120 to be affixed on skin. The present exemplary embodiment only discloses makeup as an example.

For example, when the cosmetic use is considered, the makeup information is information for makeup of part or all of a face, and means wide-ranging makeup from makeup for concealing part of spots to full makeup of the entire face. However, when uses in other fields, for example, a medical use and the like are considered, a functional material used for a region other than a face can also be used.

Furthermore, the makeup information may be provided by analyzing makeup optimized for an individual user, and may be provided as information for applying the latest makeup or makeup for a specific celebrity as an application installed in makeup information terminal 140 or as cloud service from a server via a network.

In addition, when the medical use is considered, a use for concealing a scar, such as a bruise or a burn, and a use for printing a medicinal ingredient to be used on an affected part like an adhesive plaster can be considered.

That is, use functions widely needed can be suitably added by causing a functional component needed for each use to be supported regardless of a use field.

In addition, it is possible to freely design and print by oneself a seal for cheering sports, a seal to be affixed on a face in an amusement park, and the like.

Furthermore, with makeup information terminal 140, it is also possible to analyze a user's individual beauty information from image information obtained by photographing the user's face, and to customize printing of a cosmetic agent. For example, it is also possible to have a skin-color correction function for matching a color with the user's skin color.

It is also possible to load individual face information to print an active ingredient (for example, a moisturizer, a sunscreen, a skin-whitening agent) only on a necessary place.

In addition, extraction of a characteristic point of the user's face makes it possible to print a positioning symbol or the like that assists the user in affixing the film simultaneously with a cosmetic agent, thereby assisting simpler makeup.

In printing unit 104, thin-film sheet 110 on which functional material 170 is printed is humidified by humidifier 102 before being sent to transfer unit 106. Such humidification improves softness of biocompatible thin-film 120, and lowers an adhesive force of an interface (A) between first support 130 and biocompatible thin-film 120.

Humidifier 102 facilitates and ensures transferring of thin-film printed body 160 from first support 130 to second support 150 in subsequent transfer unit 106.

Then, humidified thin-film sheet 110 on which functional material 170 is printed is sent to transfer unit 106. Here, thin-film printed body 160 with functional material 170 printed on biocompatible thin-film 120 is transferred to second support 150 supplied from outside of printing device 100 in advance by second support feeder 105 such that only a thin-film printed body 160 part comes into contact.

Operations of transferring will be described with reference to FIG. 4A to FIG. 4E.

In transfer unit 106, second support 150 is brought into intimate contact with a functional material 170 side of thin-film printed body 160 on first support 130.

Here, an adhesive force A and an adhesive force B are set to satisfy a relationship of A<B in advance, the adhesive force A being at an interface (A) between first support 130 and biocompatible thin-film 120, the adhesive force B being at an interface (B) between a surface of biocompatible thin-film 120 on which makeup information is printed and second support 150. Accordingly, thin-film printed body 160 separates from first support 130 on a biocompatible thin-film 120 side, and comes into intimate contact with second support 150 on a functional material 170 side. As a result, thin-film printed body 160 is transferred well from first support 130 to second support 150.

Then, thin-film printed body 160, while being supported by second support 150, is delivered to outside of printing device 100 by thin-film printed body delivery unit 107.

Here, an adhesive force B and an adhesive force C are set to satisfy a relationship of B<C in advance, the adhesive force B being at an interface (B) between second support 150 and thin-film 120 on which makeup information is printed, the adhesive force C being at an interface (C) between a human body (skin) and biocompatible thin-film 120. Accordingly, thin-film printed body 160 separates from second support 150 on a functional material 170 side, and comes into intimate contact with the skin on a biocompatible thin-film 120 side. As a result, thin-film printed body 160 is transferred well from second support 150 to the skin. A magnitude relationship of the adhesive forces can be evaluated by a quantitative evaluation using devices, such as a rheometer and a bondtester, and by visually determining a proportion of thin-film printed body 160 being transferred when thin-film printed body 160 is affixed on a base material or the skin.

In this way, thin-film printed body 160 that is biocompatible thin-film 120 on which functional material 170 is printed is transferred to the skin, and the makeup is completed.

Thin-film printed body delivery unit 107 not only delivers thin-film printed body 160 of a single sheet, but also may be configured as a detachable module that is a transfer device for enabling direct transfer from the module to the skin.

Finally, a method of printing a functional material on a biocompatible thin-film with the aforementioned device will be described.

This method includes:

(1) a thin-film sheet feeding step of feeding a thin-film sheet including a biocompatible thin-film and a first support into inside of a device for printing a functional material on the biocompatible thin-film,

(2) a step of loading makeup information for printing the functional material on the biocompatible thin-film,

(3) a printing step of printing a functional material corresponding to the makeup information on the biocompatible thin-film based on the makeup information from a controller,

(4) a second support feeding step of feeding a second support into inside of the device for printing the functional material on the biocompatible thin-film,

(5) a transfer step of transferring a thin-film printed body that is the biocompatible thin-film on which the functional material is printed by a printing unit from the first support to the second support, and

(6) a thin-film printed body delivery step of delivering the thin-film printed body transferred to the second support to outside of the device for printing the functional material on the biocompatible thin-film.

Here, a humidification step of humidifying the thin-film sheet may be added before or after the printing step of (3). Furthermore, the aforementioned method may include a cutting step of cutting biocompatible thin-film 120 on which functional material 170 is printed into a desired shape before the thin-film printed body delivery step of (6).

It will be appreciated from the foregoing description that the configuration of printing device 100 of the present exemplary embodiment makes it possible to easily obtain thin-film printed body 160 for assisting makeup.

The present exemplary embodiment eliminates a concern about an influence of a printing agent on a living body because a printing surface of functional material 170 of thin-film printed body 160 is always on an opposite side of a surface on a side that is to be transferred to skin. That is, since only a biocompatible thin-film side is transferred to the human body, a user having skin weak to functional material 170, such as a cosmetic agent, can also apply makeup easily and freely without limitation to a cosmetic agent or the like. When low-load thin-film printed body 160 is transferred onto a bruise until a complete cure of the bruise that remains after treatment, thin-film printed body 160 will become an item for a user who worries the bruise by treatment to live daily life with a rich spirit.

Furthermore, since thin-film sheet 110 is humidified before or after printing of functional material 170, thin-film printed body 160 can be peeled off from first support 130 with a small force during transferring from first support 130 to second support 150. Therefore, it is possible to perform transferring in transfer unit 106 securely and safely, thereby allowing printing on a thin, delicate thin-film and secure handling until transfer to the skin.

It is needless to say that the present disclosure is not limited to the aforementioned exemplary embodiment and that various modifications may be made appropriately within the intended scope of the present disclosure.

The configuration of the present disclosure makes it possible to easily apply makeup customized for an individual or the latest makeup to a human body by printing a functional material based on makeup information, such as a cosmetic agent, on a biocompatible thin-film medium, and by transferring the thin-film medium to the human body.

This enables people who do not have sufficient knowledge or techniques about makeup and people who cannot take time for makeup to apply beautiful, safe makeup easily and quickly without assistance.

1. A device for printing a functional material on a biocompatible thin-film, the device comprising:
a thin-film sheet feeder configured to feed a thin-film sheet including the biocompatible thin-film and a first support into inside of the device for printing the functional material on the biocompatible thin-film;
a controller configured to load makeup information for printing the functional material on the biocompatible thin-film;
a printing unit configured to print the functional material corresponding to the makeup information on the biocompatible thin-film based on the makeup information from the controller;
a second support feeder configured to feed a second support into inside of the device for printing the functional material on the biocompatible thin-film;
a transfer unit configured to transfer a thin-film printed body that is the biocompatible thin-film on which the functional material is printed by the printing unit from the first support to the second support; and
a thin-film printed body delivery unit configured to deliver the thin-film printed body transferred to the second support to outside of the device for printing the functional material on the biocompatible thin-film.

2. The device for printing a functional material on a biocompatible thin-film according to claim 1, wherein the thin-film sheet feeder feeds the thin-film sheet into inside of the device by rotating a roller that comes into contact with only the first support.

3. The device for printing a functional material on a biocompatible thin-film according to claim 1, further comprising:
a humidifier configured to humidify the thin-film sheet.

4. The device for printing a functional material on a biocompatible thin-film according to claim 3, wherein the humidifier sprays one of vapor and water-soluble liquid to humidify the thin-film sheet.

5. The device for printing a functional material on a biocompatible thin-film according to claim 3, wherein the humidifier humidifies the thin-film sheet by putting the thin-film sheet in one of water and water-soluble liquid.

6. The device for printing a functional material on a biocompatible thin-film according to claim 3, wherein the humidifier is provided downstream of the printing unit.

7. The device for printing a functional material on a biocompatible thin-film according to claim 1, wherein the printing unit prints the functional material on part of the biocompatible thin-film based on user information.

8. The device for printing a functional material on a biocompatible thin-film according to claim 7, wherein the printing unit prints a symbol for simplifying transfer on part of the biocompatible thin-film.

9. The device for printing a functional material on a biocompatible thin-film according to claim 1, wherein the transfer unit transfers the thin-film printed body such that a surface of the thin-film printed body on which the functional material is printed comes into contact with the second support.

10. The device for printing a functional material on a biocompatible thin-film according to claim 1, wherein, in the transfer unit, an adhesive force A and an adhesive force B satisfy a relationship of A<B, the adhesive force A being at an interface between the first support and the thin-film, the adhesive force B being at an interface between the thin-film surface on which the makeup information is printed and the second support.

11. The device for printing a functional material on a biocompatible thin-film according to claim 1, wherein, in the transfer unit, an adhesive force B and an adhesive force C satisfy a relationship of B<C, the adhesive force B being at an interface between the thin-film surface on which the makeup information is printed and the second support, the adhesive force C being at an interface between the thin-film and skin to which the thin-film printed body is transferred.

12. The device for printing a functional material on a biocompatible thin-film according to claim 1, further comprising:
a cutting unit configured to cut one of the thin-film and the thin-film printed body into a desired shape.

13. The device for printing a functional material on a biocompatible thin-film according to claim 1, wherein the makeup information includes a skin-color correction function optimized for a skin color of each user.

14. The device for printing a functional material on a biocompatible thin-film according to claim 1, further comprising:
a tank for supplying the functional material to be printed on the biocompatible thin-film.

15. The device for printing a functional material on a biocompatible thin-film according to claim 1, further comprising:
a ribbon for retaining the functional material to be transferred to the biocompatible thin-film.

16. A method of printing a functional material on a biocompatible thin-film, the method comprising:
a thin-film sheet feeding step of feeding a thin-film sheet including the biocompatible thin-film and a first support into inside of a device for printing the functional material on the biocompatible thin-film;
a step of loading makeup information for printing the functional material on the biocompatible thin-film;
a printing step of printing the functional material corresponding to the makeup information on the biocompatible thin-film based on the makeup information from a controller;
a second support feeding step of feeding a second support into inside of the device for printing the functional material on the biocompatible thin-film;
a transfer step of transferring a thin-film printed body that is the biocompatible thin-film on which the functional material is printed by a printing unit from the first support to the second support; and
a thin-film printed body delivery step of delivering the thin-film printed body transferred to the second support to outside of the device for printing the functional material on the biocompatible thin-film.

17. The device for printing a functional material on a biocompatible thin-film according to claim 2, further comprising:
a humidifier configured to humidify the thin-film sheet.

18. The device for printing a functional material on a biocompatible thin-film according to claim 17, wherein the humidifier sprays one of vapor and water-soluble liquid to humidify the thin-film sheet.

19. The device for printing a functional material on a biocompatible thin-film according to claim 17, wherein the humidifier humidifies the thin-film sheet by putting the thin-film sheet in one of water and water-soluble liquid.

20. The device for printing a functional material on a biocompatible thin-film according to claim 17, wherein the humidifier is provided downstream of the printing unit.
